(19) United States
(12) Patent Application Publication
(10) Pub. No.: US 2002/0064791 A1
(43) Pub. Date: May 30, 2002

(54) Detection of unlabeled hybridized DNA and RNA using restriction enzyme digestion

(76) Inventors: Tom J. Whitaker, Knoxville, TN (US); Wanda L. B. White, Oak Ridge, TN (US); K. Bruce Jacobson, Oak Ridge, TN (US); Kenneth F. Willey, Schwenksville, PA (US); Bruce M. Applegate, West Lafayette, IN (US)

Correspondence Address: BIRCH STEWART KOLASCH & BIRCH, PO BOX 747, FALLS CHURCH, VA, 22040-0747, US

(21) Appl. No.: 09/955,037

(22) Filed: Sep. 19, 2001

Related U.S. Application Data
(60) Provisional application No. 60/233,486, filed on Sep. 19, 2000.

(51) Int. Cl. C12Q 1/68; C07H 21/04

(52) U.S. Cl. 435/6; 536/24.3

(57) ABSTRACT

This invention provides a means of detecting unlabeled DNA or RNA following hybridization to an immobilized, labeled DNA probe. The immobilized DNA oligomers (probes) form a hairpin structure containing a unique restriction site that persists only when hybridization to the internal target-hybridization sequence has not occurred. Restriction enzyme digestion of unhybridized, labeled DNA probes at or above room temperature detaches the label from the surface and the label is washed away. The hairpin structure is disrupted when hybridization of the internal target-hybridization sequence occurs, removing the restriction site and preventing cleavage. In this case, the labels on target-hybridized probes remain bound to the substrate and are detected. In its preferred embodiment, a bioluminescent label is located on one end of the probe and a surface attachment moiety is on the other. Applications include characterization of mutations and single nucleotide polymorphisms in DNA and RNA, genomic fingerprinting, analysis of DNA or RNA sequence for medical diagnostics, and pathogen detection and identification.

### TECHNICAL FIELD

This invention is related to the fields of deoxyribonucleic acid (DNA) and ribonucleic acid (RNA) sequencing; and genetic mapping, characterization, and diagnostics; and detection and identification of organisms, viruses, and nucleic acids.

### BACKGROUND OF THE INVENTION

DNA and RNA—The nuclei of living cells possess chromosomes, which contain the genetic information necessary for growth, regeneration and other functions of organisms. Instructions concerning such functions are contained in the molecules of deoxyribonucleic acid (DNA). The genetic information of DNA is contained in the sequence of nucleotide bases, which are arranged on a linear polymer of deoxyribose phosphates. The four bases are thymine (T), adenine (A), cytosine (C), and guanine (G). Two strands of DNA form a double helix joined by base pairing of Ts to As and Cs to Gs. Accordingly, the base sequence along one strand determines the order of bases along the complementary strand. Cells use DNA as a template for RNA synthesis. During RNA synthesis, a strand of RNA complementary to the template DNA is formed. RNA/DNA hybrids obey the same base pairing rules as DNA/DNA hybrids except that RNA contains uracil (U) instead of T. Both RNA and DNA, therefore, can be used to derive genetic information.

DNA and RNA Sequencing—Sequencing is the determination of the sequence of bases in nucleic acid strands. Polymerase chain reaction (PCR) is a process in which segments of sample DNA that are of interest are synthesized repeatedly until an amount is produced which is suitable for further experimentation. For the purpose of sequencing, PCR can contain fluorescently labeled, dideoxynucleotide triphosphates that stop further elongation of strands. Each of the four kinds of dideoxynucleotide triphosphates (A, T, C, or G) is labeled with a different fluorescent label. The products of this reaction are of various lengths and each strand has only one label that represents the last base added. When separated by size, the base sequence of the template DNA can be determined. This is the Sanger method of DNA sequencing and is, at present, the most commonly used method for sequencing long DNA sequences. For RNA, PCR includes reverse transcriptase that makes a complementary DNA from the RNA template. The DNA products are then sequenced by the Sanger method. The Sanger method requires a high quality gel and electrophoresis of the ampified DNA. It is a time consuming process and not well-suited for parallel sequencing of several sequences.

Sequencing by Hybridization—Sequencing by hybridization (SBH) uses the specificity of DNA/DNA binding rules and melting temperature to sequence labeled sample DNA. Hybridization is performed at elevated temperatures close to the melting point of the fully complementary hybrid so that non-complementary DNA cannot hybridize to the probe. Also, imperfect hybridization can be followed by washes approaching the melting temperature to ensure that non-complementary sample DNA has been removed from the probe site. Typically, probes are immobilized on a solid surface. Detection of labeled DNA at the location of a probe indicates the presence of complementary sequence in the sample DNA. The solid surface containing the probe oligonucleotides is sometimes referred to as an SBH chip, a genosensor chip, a hybridization surface, or a dot blot. This version of the SBH process is referred to as Format II SBH. E. Southern disclosed Format II SBH in International Application No. PCT/GB89/00460.

Format I SBH is an alternative method where sample DNA is attached to a solid surface, such as a nylon membrane, and hybridized with labeled probes. R. Drmanac and R. Czerkvenjakov disclosed Format I SBH in U.S. Pat. No. 5,202,231.

Genetic diagnostic information can also be obtained using techniques similar to those used for obtaining sequencing information. In diagnostic applications, a known sequence that indicates the presence of a particular gene or organism is immobilized to the surface and labeled DNA from the sample is allowed to hybridize. After washing to eliminate nonspecific binding, detection of the label indicates that gene or organism is present in the sample.

While the above sequencing by hybridization techniques are capable of producing reliable results, if properly applied, certain disadvantages are inherent. For example, the target DNA must be sorted and immobilized (Format I) or labeled (Format II). These procedures for sequencing are laborious and require a large DNA or RNA sample. If these limitations can be overcome, the parallel nature of the genosensor chip makes it ideal for several forms of diagnostics, forensics, and other applications. In particular, if amplification, labeling, and segregation of the target DNA before hybridization were not necessary, the genosensor chip could be used more readily as a routine diagnostic test. DNA arrays utilize parallel hybridization of target DNA to multiple known-sequence probes in a compact array. This allows detection of multiple sequences using SBH with only one small DNA sample [1-8]. Since the arrays are so compact (e.g., 100s to 1000s on a silicon chip), the reliability of diagnostic tests can be improved by using multiple probes.

Probe Technology—Molecular beacons (described in U.S. Pat. Nos. 5,118,801 and 5,312,728) are single-stranded DNA (ss-DNA) probes that form a stem-and-loop structure. The loop sequence is complementary to a sequence of interest. Binding of complementary DNA to the loop sequence opens the stem-and-loop structure. If no complementary DNA is present, the stem-and-loop structure remains intact. Molecular beacons do not require labeling of the sample DNA because the beacons have a single fluorophore attached to one end and a quencher to the other end. When in a stem-and-loop conformation, the ends are close together and light emission is quenched. When hybridization breaks the stem structure, the fluorophore and quencher are separated and light is emitted [9]. Although the stem is only 6 or 7 base pairs in length, it has a melting temperature well above 55° C. due to its intramolecular nature. Molecular beacons are routinely used to monitor PCR reactions in real time.

Molecular beacons can be used to differentiate sequences that differ by only a single base such as alleles for drug-resistant tuberculosis, human coagulation factor V, human estrogen receptor, and human methylenetetrahydrofolate reductase [10-13]. This specificity is not available with most other probe types. In addition to DNA studies, molecular beacons have been used to detect RNA sequences in living cells [14-16]. It has been demonstrated that molecular beacons still function when immobilized to surfaces [17-18].

It is necessary that molecular beacons form the desired stem-and-loop structure. One useful tool is a DNA folding program available on the internet (http://bioinfo.math.rpi.edu/˜zukerm/) that estimates the free energy of formation of the stem hybrid and predicts the melting temperature. In general, the loop areas should be approximately 21 bases long with a GC content of 25-75 percent. The loop length can be adjusted to accommodate various GC percentages. The stem structures should be 6 or 7 base pairs long and contain mainly Cs and Gs.

Although molecular beacons have many good qualities, they are limited for use in applications other than PCR diagnostics. The use of a single fluorophore label requires the amplification of the sample DNA. Because many other compounds fluoresce and produce a high background, a large amount of fluorophore must be present to be measured accurately.

Label Technology—Bioluminescent enzymes, such as luciferase, alkaline phosphatase, horseradish peroxidase, β-galactosidase, and aequorin, have recently been used to detect attomolar concentrations of molecules and have been shown to be 30-60 fold more sensitive than radioactive labels [19]. They produce much less background since no excitation light is required. In these particular cases, light is produced continuously as long as activator is available, therefore, photons can be counted over a period of time. Label technology is a rapidly advancing field, and, for this reason, a type of probe that is easily adaptable to new labels or multiple labels would be highly beneficial.

Restriction enzymes—Restriction enzymes (or restriction endonucleases) are produced in bacteria, presumably to degrade foreign DNA. Methylation differences between the bacterium's genomic DNA and the foreign DNA protect the genomic DNA from cleavage [20].

Restriction enzymes bind at recognition sequences. Recognition sequences are typically 4 to 6 bases long, but may be longer. Restriction enzymes cleave double-stranded DNA (dsDNA) at a restriction site, which may or may not be located within the recognition sequence. At each restriction site, one phosphodiester bond from each of the strands in the dsDNA is hydrolyzed to form hydroxyl and phosphate groups. The cleaved sites, one on each DNA strand, may be opposite each other forming two blunt-ended dsDNA fragments, or may occur at different locations resulting in fragments with protruding unpaired bases called sticky ends [21].

Currently, almost 3000 restriction enzymes with over 200 different recognition sequences are known. The field of molecular cloning relies heavily on the use of restriction enzymes to create new chimeric DNA sequences from different sources [22]. This demand is met by several companies (New England Biolabs, Fermentas, Roche, and Stratagene, for example) that offer restriction enzymes and study the activities of the enzymes. Restriction enzymes are usually supplied with a concentrated buffer solution that optimizes activity. Typical buffer conditions are pHs from 7.0 to 7.9, magnesium salts from 10 to 100 mM, bovine serum albumin from 0 to 100 μg/ml, and temperatures from 30 to 65° C. One unit of restriction enzyme is defined as the amount necessary to digest 1 μg of DNA in one hour under optimal conditions.

The most commonly used restriction enzymes are highly efficient (nearly 100% of restriction sites cut). DNA fragment length can affect restriction enzyme activity. Frequently, restriction enzymes can cut short pieces of dsDNA efficiently, but some require longer fragments. Suppliers (New England Biolabs, for example) recommend having six base pairs on each side of recognition sites to ensure efficient cleavage when length preference is unknown. Some enzymes, such as Eco RI, are known to cut efficiently with only one base on each side of the recognition site.

### SUMMARY OF INVENTION

The present invention utilizes a nucleic acid probe for detecting specific target nucleic acid sequences. The probe is an oligonucleotide containing a covalent surface-coupling group attached to one end and a label attached to the other end, wherein the oligonucleotide contains two complementary regions which hybridize to each other in the absence of target nucleic acid forming a loop region which contains a sequence complementary to the target sequence being probed, and forming a stem region which contains a restriction enzyme site which is not present in the hybridized loop.

The present invention further utilizes a method of determining the presence of one or more specific target nucleic acid sequences using such a probe.

### BRIEF DESCRIPTION OF THE DRAWINGS

FIG. 1. The structure of a probe with a removable label (PRL) attached to a glass surface. G, C, A, and T represent the bases of nucleotides in an ssDNA molecule. The looped area contains the target-hybridization sequence. Stem-forming sequences, located on each side of the target-hybridization sequence, hydrogen bond to form the stem. This stem can be cut by the restriction enzyme Pal I.

FIG. 2. A summary of the processes in a preferred embodiment.

### DETAILED DESCRIPTION OF THE INVENTION

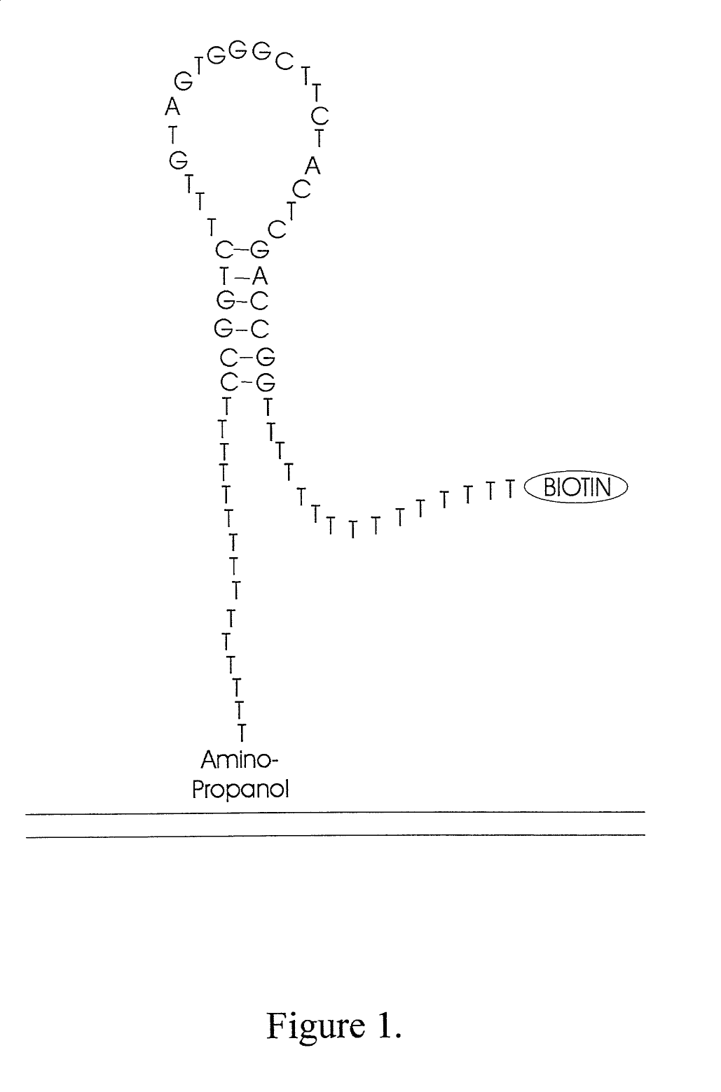

The invention is a probe with a removable label (PRL) that can be used to detect hybridization without labeling target DNA. The PRL structure differs from that of a molecular beacon in three ways: 1) the stem structure contains a restriction enzyme recognition site, 2) different label types, including fluorescent labels, can be used and no quencher is necessary, 3) a moiety is present to attach the PRL to surfaces to form arrays (FIG. 1). Like molecular beacons, the stem structure in the PRL must open upon hybridization of the loop sequence to complementary target DNA or RNA. The stem structures may be approximately 4-9 nucleotides in length. For each PRL array, a restriction enzyme is used that has a restriction site in each stem sequence but does not have a restriction site or a recognition site in any of the hybridized loop sequences. A list of several potential restriction enzymes and their recognition sequences is given in Table 1. Identification of additional suitable restriction enzymes can be readily determined based on the present disclosure of the invention and generally available knowledge in the field of molecular biology. For example, the PRL shown in FIG. 1 has both the Pal I recognition site and restriction site located in the stem. The stem sequence GGCCAG, written 5′ to 3′, can be cut by Pal I forming the fragments GG and CCAG. After this fragmentation, covalent bonds no longer connect the label to the surface. The six weaker hydrogen bonds between the two CG base pairs are not strong enough to hold the label to the bound portion of the PRL and the label can be washed away. Other stem sequences, used with other restriction enzymes in Table 1, may have more than six hydrogen bonds remaining. Washes at elevated temperatures can be used to break these hydrogen bonds without damaging the covalent bonds. The conditions used for washing away the labeled nucleotide fragment subsequent to restriction enzyme digestion may be routinely determined by one skilled in the art based on the restriction site and restriction enzyme used. Such information is typically available from the company source of the restriction enzyme.

The loop portion of the PRL should be about 16 to 25 nucleotides in length. In order to function properly, the hybrid formed upon hybridization of a fully complementary DNA to the loop sequence must be more energetically favorable than the stem hybrid, however, any loop hybrid containing mismatches must be energetically less favorable than the stem hybrid. In order to achieve this, the length or composition of the loop and/or stem sequences is varied. Commonly, a loop sequence with a high AT content requires more nucleotides in the loop sequence or a weaker stem sequence. A loop sequence with a higher GC content may need a reduced length or a more stable stem sequence.

TABLE 1
A list of restriction enzymes and their recognition sequences. Abbreviations are as
follows: A, indicates the cleavage site; _, indicates the second cleavage site in “sticky end”
cleavages; N, any of the four bases; W, base A or T; M, base A or C; K, base G or I ; Y,
base C or T; R, base G or A; S base G or C; D base A or G or T; II base A or C or T. Table
adapted from the Restriction Enzyme Database (http://rebase.neb.com), Dr. Richard J.
Roberts and Dana Macelis, authors.

| Enzymes | Recognition Sequence | Other Enzymes Capable of Cutting These Sequences |
|---|---|---|
| AluI | AGˆ CT | MitI |
| BalI | TGGˆ CCA | MlsI, Mlu31I, MIuNI, MscI |
| BsaAI | YACˆ GTR | BstBAI, MspYI, PsuAI |
| BsrBI | CCGˆ CTC | AccBSI, BstD102I, Bst31NI, MbiI |
| BtrI | CACˆ GTC | — |
| Cac8I | GCNˆ NGC | — |
| CviJI | RGˆ CY | CviTI |
| CvIRI | TGˆ CA | HpyCH4V |
| Eco47III | AGCˆ GCT | AfeI, AitI, Aor51HI, FunI |
| Eco7SI | GGCˆ GCC | EgeI, EheI, SfoI |
| EcoICRI | GAGˆ CTC | EcIJ361I, Eco53kI, kfraI |
| FnuDII | CGˆ CG | AccII, BceBI, BepI, Bpu95I, Bsh1236I, Bsp50I, Bsp123I, |
| | | BstFNI, BstUI, Bsu15321, BtkI, Csp68KVI, CspKVI, FauBlI, |
| | | MvnI, ThaI |
| HaeI | WGGˆ CCW | — |
| HaeIII | GGˆ CC | BecAII, Bim19II, Bme361I, BseQI, BshI, BshFI, Bsp211I, |
| | | BspBRI, BspKI, BspRI, BsuRI, BteI, CltI, DsaII, FnuDI, |
| | | MchAII, MfoAI, NgoPII, NspLKI, PalI, Pde133I, PfKI, PlaI, |
| | | SbvI, SfaI, SuaI |
| Hindu | GTYˆ RAC | HinJCI, HincII |
| Hpy8I | GTNˆ NAC | HpyBII |
| LpnI | RGCˆ GCY | Bme142I |
| MstI | TGCˆ GCA | Accl6I, AosI, AviII, FdiII, EspI, NsbI, PamI |
| NaeI | GCCˆ GGC | CcoI, PdiI, SauBMKI, SauHPI, SauLPI, SauNI, SauSI, |
| | | Slu1777I |
| NIaIV | GGNˆ NCC | AspNI, BscBI, BspLI, PspN4I |
| NruI | TCGˆ CGA | Bsp68I, MZuB2I, Sbo13I, SpoI |
| NspBII | CMGˆ CKG | MspAlI |
| PmaCI | CACˆ GTG | BbrPI, BcoAI, Eco72I, PmiI |
| PvuII | CAGˆ CTG | BaA, BavAI, BavBI, Bspl53AI, BspM39I, Bsp04I, Cfr6I, |
| | | DmaI, Ecu, NmeRI, Pae17kI, Pvu84II |
| RsaI | GTˆ AC | AfaI, HpyBI, PlaAII |
| SelI | CTCˆ GAG | — |
| SmaI | CCCˆ GGG | CfrJ4I, PaeBI, PspALI |
| SrfI | GCCCˆ GGGC | — |
| SspI | AATˆ ATT | — |
| StuI | AGGˆ CCT | AatI, AspMI, Eco147I, GdzI, Pme55I, SarI, Sru30DI, SseBI, |
| | | SteI |

Type II restriction enzymes cleaving outside their recognition sequences:

| | | |
|---|---|---|
| BsrI | ACTG_GNˆ | Bse1I, BseNI, BsrSI, Bstl1I, Tsp1I |
| BstF5I | GGATG_NNˆ | BseGI |
| BtsI | GCAGTG_NNˆ | — |

Type II restriction enzymes producing 5′ overhangs:

| | | |
|---|---|---|
| AccI | GTˆ MK_AC | FblI, XmiI |
| AciI | Cˆ CG_C | — |
| AcyI | GRˆ CG_YC | AhalI, Aash, AstWI, AsuIII, BbiII, BsaHI, BstACI, HgiI, |
| | | HgiDI, HgiGI, HgiHII, HinlI, Hsp92I, Msp17I, PamiI |
| AflIII | Aˆ CRYG_T | — |
| AgeI | Aˆ CCGG_T | AsiAI, BshTI, CspAI, PinAI |
| ApaLI | Gˆ TGCA_C | Alw44I, SnoI, VneI |
| AscI | GGˆ CGCG_CC | — |
| Asp718I | Gˆ GTAC_C | Acc65I, AhaB8I, SthI |
| AsuI | Gˆ GNC_C | AspS9I, AvcI, Bac36I, Bal228I, BavAII, BavBII, Bce22I, |
| | | BshKI, BsiZI, Bsp1894I, BspBII, BspF4I, Bsu54I, CcuI, |
| | | Cfrl3I, MaeK8TlI, NspIV, Nsp7l2lI, Pdel2I, PspPI, Sau96I |
| AsuII | TTˆ CG_AA | AcpI, Asp10HI, BimI, Bim19I, Bpu14I, BsiCI, Bsp1191, |
| | | BspLAII, BspTlO4I, BstBI, CbiI, Csp45I, Csp68KlI, FspII, |
| | | LspI, MlaI, NspV, PlalI, PpaAI, SfuI, SsplI, SspRFI, SviI |
| AvaI | Cˆ YCGR_G | Ama87I, AquI, BcoI, Bse15I, BsoBI, BstSI, Eco88I, Eco27kI, |
| | | NspIII, NspSAI, PlaAI, PunAI |
| AvaII | Gˆ GWC_C | AflI, Asp7451, BamNxI, BcuAI, Bme18I, Bme216I, BsrAI, |
| | | BthAI, CauI, Csp68KI, DsaIV, EagMI, Eco47I, ErpI, FdiI, |
| | | FspMSI, FssI, HgiBI, HgiCII, HgiEI, HgiHIII, HgiJI, Kzo49I, |
| | | SinI, SmuEI, VpaK11BI, |
| AvrII | Cˆ CTAG_G | AvrBII, BinI, BspA2I, XmaJI |
| BamHI | Gˆ GATC_C | AccEBI, AliI, ApaCI, AsiI Bce751I, BnaI, Bsp98I, Bsp4009I, |
| | | BspAAIII, BstI, CelI, Mlu23I, Nsp29132II, NspSAIV, OkrAI, |
| | | Pfl8I, RspLKII, SoiI, Sun |
| BbvCI | CCˆ TCA_GC | AbeI |
| BetI | Wˆ CCGG_W | Bca77I, BsaWI |
| Bpu10I | CCˆ TNA_GC | BpuDI |
| BsePI | Gˆ CGCG_C | BssHII, BstBZ153I, PauI |
| BsiI | Cˆ ACGA_G | BssSI, Bst2BI |
| Bsp120I | Gˆ GGCC_C | PspOMI |
| BspMIII | Tˆ CCGG_A | AccIII, Aor13HI, BbvAIII, BseAI, BsiMI, Bsp13I, BspEI, |
| | | Bsu23I CauB3I, Kpn2I, MroI, PinBII, PtaI |
| BstNi | CCˆ W_GG | AeuI, AonI, ApaORiI, ApyI, BcIBII, Bse16I, Bse17I, Bse24I, |
| | | BseBI, BshGJ, BsiLI, BspNI, BstlI, Bst2I, Bst38I, Bst200I, |
| | | BstM6I, BstOI, Bst2UI, BthDI, BthEI, CbrI, CthII, Fsp1604I, |
| | | MvaI, SniI, SslI, Sthl17I, TaqXI, ZanI |
| BstEII | Gˆ GTNAC_C | AcrII, AspAI, Bse64I, BseT9I, BseTlOI, BsiKI, BstPI, BstT9I, |
| | | BstT10I, EcaI, Eci125I, Eco91I, Eco065I, Eco0128I, |
| | | NspSAII, PspEI |
| CaulI | CCˆ S_GG | AhaI, AselI, AsuC2I, BcnI, HgiS22I, Mgl14481I, NciI |
| CfrI | Yˆ GGCC_R | Bfi89I, EaeI, EcoHK31I |
| Cfr10I | Rˆ CCGG_Y | Bcol18I, Bsel18I, Bse6341, BsrFI, BssAI, BstB7SI |
| CviAII | Cˆ AT_G | — |
| CviQI | Gˆ TA_C | Csp6I, CviRII |
| DdeI | Cˆ TNA_G | BstDEI, BstJZ301I |
| DralI | RGˆ GNC_CY | EcoO109I |
| DsaI | Cˆ CRYG_G | BstDSI, BtgI, TspBI |
| EcoHI | ˆ CCSGG_{—} | Eco1831I, Kpn49kII |
| EcoRII | ˆ CCWGG_{—} | PspGI, SleI, SspAI |
| Eco56I | Gˆ ACCGG_C | MroNI, NgoAIV, NgoMIV, ScelII, SrlI |
| EspI | GCˆ TNA_GC | BipI, Bpul102I, Bsp1720I, CelII |
| Fnu4HI | GCˆ N_GC | BsoFI, Bsp6I, FbrI, Fsp4HI, ItaI, SatI, Uur96OI |
| GdiII | Cˆ GGCC_R | — |
| HgiCI | Gˆ GYRC_C | AccB1I, BanI, BbvBI, BshNI, BspT107I, Eco64I, HgiHI, |
| | | MspB4I, PfaAI |
| HinfI | Gˆ ANT_C | CviBI, FnuAI, HhaII, SscL1I |
| HinP1I | Gˆ CG_C | Hin6I, HsoI, HspAI, SciNI |
| HpaII | Cˆ CG_G | Bco27I, BsiSI, Bst40I, BsuFI, CboI, HapII, Hin2I, MnoI, |
| | | MspI, Pdel37I, Sthl34I |
| Hpyl78III | TCˆ NN_GA | Hpyl88III |
| KasI | Gˆ GCGC_C | — |
| MaeI | Cˆ TA_G | BfaI, EgoI, MthZI, RmaI, XspI |
| MaeII | Aˆ CG_T | HpyCH4IV |
| MaeIII | ˆ GTNAC_{—} | — |
| MboI | ˆ GATC_{—} | AspMDI, Bce2431, Bfi57I, Bmel2I, BscFI, Bsp67I, Bsp105J, |
| | | Bsp143I, Bsp20951, BspAI, BspFI BspJI, BstENII, BtkII, Cad, |
| | | CcyI, Cpfi, CviAI, DpnII, FnuCI, FnuEI, Had, Kzo9I, LlaAI, |
| | | MgoI, MkrAI, NdeII, NialI, NmeCI, NphI, RaIF40I, Sau3AI, |
| | | SauMI, Sth368I |
| MluI | Aˆ CGCG_T | Bbi24I |
| Nan | GGˆ CG_CC | MchI, Mlyl131, NdaI, NunII, SseAI |
| NcoI | Cˆ CATG_G | Bsp19I |
| NheI | Gˆ CTAG_C | AsuNHI, PstNHI |
| NotI | GCˆ GGCC_GC | CciNI, CspBI, MchAI |
| PpuMI | RGˆ GWC_CY | Pfl27I, PpuXI, Psp5II, PspPPI |
| RsrII | CGˆ GWC_CG | CpoI, CspI, Rsr2I |
| SaiI | Gˆ TCGA_C | BspMKI, HgiCIII, HgiDII, NopI, RflFI, RtrI, Rtr63I, XciI |
| SanDI | GGˆ GWC_CC | Sse1825I |
| SauI | CCˆ TNA_GG | AocI, AxyI, BIiHKI , Bse21I, BspR7I, Bsu36I, CvnI, Eco81I, |
| | | Lmu60I, MstII, OxaNI, SshAI |
| ScrFI | CCˆ N_GG | Bme1390I, Msp67I, MspR9I |
| SecI | Cˆ CNNG_G | BsafI, BseDI, BssECI |
| SelI | ˆ CGCG_{—} | — |
| SexAI | Aˆ CCWGG_T | MabI |
| SfeI | Cˆ TRYA_G | BdiSI, BfinI, BstSFI, LIaBI, SfcI |
| SgrAI | CRˆ CCGG_YG | — |
| SimI | GGˆ GTC_{—} | — |
| SmiI | Cˆ TYRA_G | — |
| SplI | Cˆ GTAC_G | BpuB5I, BsiWI, BvuBI, MaeK81I, Pfl23II, PpuAI, PspLI, |
| | | SunI |
| Sse232I | CGˆ CCGG_CG | — |
| SsoII | ˆ CCNGG_{—} | BssKI, BstMZ611I, DsaV, Ecl18kI, Eco13kI, Eco21kI, |
| | | Ecol37kI, Kpn2kI, StyD4I |
| StyI | Cˆ CWWG_G | BsmSI, BssTlI, CfrBI, Eco130I, EcoT14I, ErhI, ErhB9II |
| TaqI | Tˆ CG_A | PpaAII, Tsp32I, Tsp32II, TthHB8I |
| TheI | Gˆ CWG_C | AceI, Taq52I |
| Tsp45I | ˆ GTSAC_{—} | Hpy51I, NmuCI |
| UnbI | ˆ GGNCC_{—} | — |
| VpaK1IAI | ˆ GGWCC_{—} | — |
| XhoI | Cˆ TCGA_G | AbrI, BIuI, BspAAI, BssHI, BstVI, CcrI, MavI, |
| | | PaeR7I, PanI, Sau32391, SauLPiI, 8bi681, Sfr274I, SlaI, |
| | | Sal10179I, StrI, TliI, XpaI |
| XholI | Rˆ GATC_Y | BloHI, BstX2I, BstYI, DsaIII, MfilL, PsuI, Tru201I |
| XmaI | Cˆ CCGG_G | AhyI, Cfr9I, EaeAI, EclRI, Pac25I, PspAI, XcyI, XmaCI |
| XmaIII | Cˆ GGCC_G | AaaI, BseX3I, BstZI, EagI, EcZXI, Eco52I, SenPTL6I |

Type II restriction enzymes producing 3′ overhangs:

| | | |
|---|---|---|
| AatlI | G_ACGTˆ C | Ssp5230I |
| AcelI | G_CTAGˆ C | — |
| APaI | G_GGCCˆ C | PpeI |
| BbeI | G_GCGCˆ C | — |
| BseSI | G_KGCMˆ C | — |
| BspKT6I | G_ATˆ C | — |
| BsrI | ACTG_GNˆ | BselI, BseNI, BsrSI, Bst21I, Tsp1I |
| BstF5I | GGATG_NNˆ | BseGI |
| BstXI | CCAN_NNNNˆ NTGG | BstHZ55I |
| ChaI | _GATCˆ | — |
| FmuI | G_GNCˆ C | — |
| FseI | GG_CCGGˆ CC | — |
| HaeII | R_GCGCˆ Y | AccB2I, Bspl43II, BstH2I |
| HgiAI | G_WGCWˆ C | Alw2lI, AspHI, Bbvl2I, Bsh45I, BsiHKAI, MspV28lI |
| HgiJII | G_RGCYˆ C | BanII, BpuI, Bsp519I, Bsu1854I, BvuI, Eco24I, Eco75KI, |
| | | EcoT38I, Fri0I, KoxII, PaeHI, SacNI |
| HhaI | G_CGˆ C | AspLEI, BspLAI, BstHHI, CfoI, FnuDlII |
| Hpy99I | J_CGWCGˆ | — |
| Hpy188I | TC_Nˆ GA | — |
| HpyCH4I | C_ATˆ G | — |
| KpnI | G_GTACˆ C | — |
| McrI | CG_RYˆ CG | BsaOI, Bsh1285I, BsiEI, BstMCI |
| NlaIII | _CATGˆ | HinliI, Hsp92II |
| Nli3877I | C_YCGRˆ G | — |
| NspI | R_CATGˆ Y | BstNSI, NspHI, PauAI, PunAII, XceI |
| PspO3I | G_GWCˆ C | — |
| PssI | RG_GNCˆ CY | Kaz48kI |
| PstI | C_TGCAˆ G | AloI, AliAJI, ApiI, Asp7l3I, Bsp63I, BspBI, BsuBI, CflI, |
| | | CfrA4I, CfuII, CstI, Ecl37kI, Ecl2zI, Ha/LI, MhaAI, PaePI, |
| | | P1721I, Psp23I, SalPI, SflI, YenI |
| PvuI | CG_ATˆ CG | Afa22MI, Afal6RI, BspCI, EagBI, ErhB9I, MvrI, NblI, Pie19I |
| | | Psul61I, RshI, XorII |
| SacI | G_AGCTˆ C | Psp124BI, SstI |
| SacII | CC_GCˆ GG | Cfr42I, CscI, Eae46I, Eco29kI, GalI, GceI, GceGLI, Kpn378I, |
| | | KspI, NgoAIII, NgoPIII, PaeAI, PaeQI, PaeSkI, Pael4kI, |
| | | SchZI, SenPT14bI, SexBI, SexCI, Sfr303I, SgrBI, SpuI, SstII |
| SduJ | G_DGCHˆ C | AocII, BmyI, BsoCI, Bsp 12861, BspLS2I, MblI, NspII |
| SglI | GCG_ATˆ CGC | AsiSI |
| SphI | G_CATGˆ C | BlmI, PaeI, PfaAIII, RspLKI, SpaHI |
| Sse8387I | CC_TGCAˆ GG | SbfI, SdaI |
| TaiI | _ACGTˆ | TscI, Tsp49I |
| TauI | G_CSGˆ C | — |
| Tsp4CI | AC_Nˆ GT | Bst4CI, HpyCH4III, TaaI |

In the preferred embodiment shown in FIG. 1, an aminopropanol moiety is at the 3′ end of the PRL. This group can be used to covalently attach DNA oligonucleotides to glass surfaces and form arrays [23]. A biotin moiety is bound to the 5′ end of the PRL (FIG. 1). Biotin and streptavidin have a strong affinity for each other and are routinely used as stable linking agents. Bioluminescent enzymes can be conjugated to streptavidin. When streptavidin-enzyme conjugate and biotin-labeled PRLs are incubated together, the bioluminescent enzyme becomes attached to the PRLs. Polythymine nucleotide spacers are located at the ends of the PRL (FIG. 1). The number of thymine nucleotides in these spacers can be adjusted to decrease steric hindrances of restriction enzyme activity. Other organic moieties, such as alkane chains, could be used as spacers.

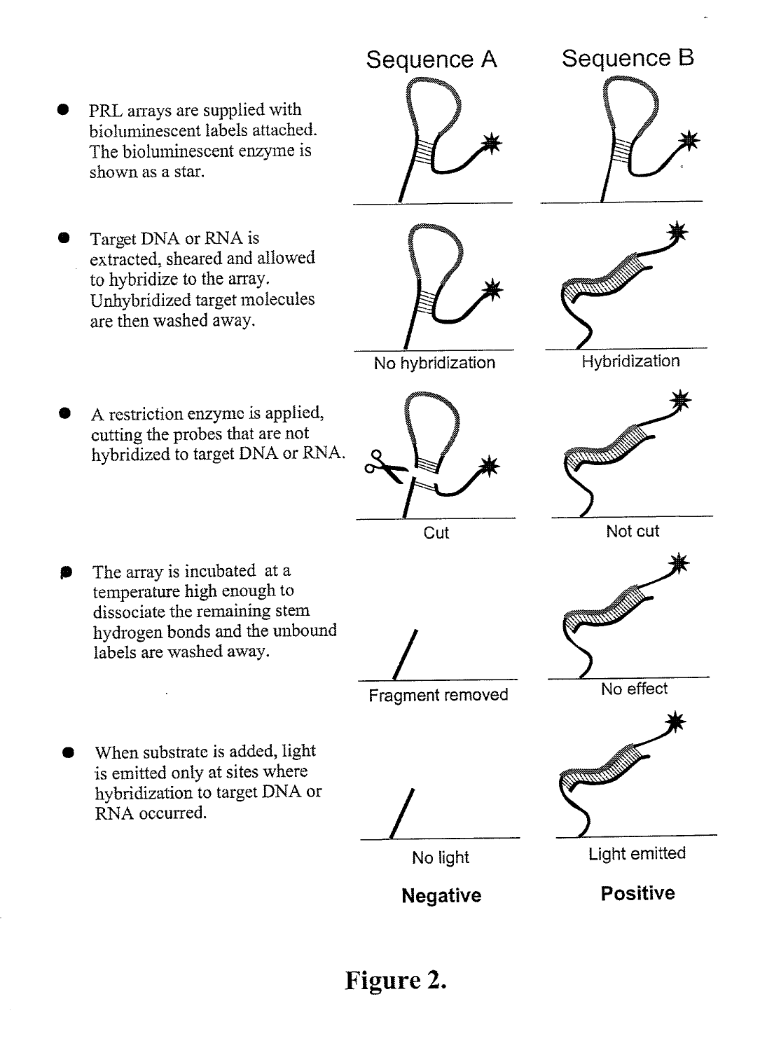

This embodiment demonstrates the utility of this invention for determining the presence or absence of specific RNA or DNA sequences (FIG. 2). The invention will result in a product that could be supplied as an array of bound, bioluminescently-labeled PRLs. Each PRL would have a stem structure where two complementary regions of the same molecule hybridize. The array will have the streptavidin-bioluminescent enzyme conjugate attached to the PRLs. DNA and/or RNA will be purified by a clinician according to standard methods, which are available in several nucleic acid purification kits. The purified nucleic acids may be sheared into smaller pieces suitable for hybridization and incubated in a buffered solution with the PRL array for hybridization to occur. The temperature and conditions of the hybridization step will be determined by the stability of the possible individual hybrids in the PRL array. A temperature that reduces non-specific binding of target sequences that are not completely complementary to the array loop sequences can be determined for each array of PRLs. After hybridization occurs, the array will be washed and restriction enzyme buffer and restriction enzyme will be added. During incubation with the restriction enzyme solution, the PRLs that are in a hairpin configuration will be cut inside the stem sequence. This cleavage will leave only a few hydrogen bonds holding the label to the surface. The bonds will be unstable and break at room temperature, releasing the label from the bound end of PRL. The array will then be washed again to remove the unbound labels and restriction enzyme. The remaining bound bioluminescent enzyme labels will be detected by incubating the array with the appropriate substrate solution and measuring photon production. Photon production at a probe site signifies that the sequence within the loop portion of the probe is complementary to a sequence in the target DNA or RNA. Lack of photon production at a PRL sight indicates the absence of a sequence in the target DNA or RNA that is complementary to the probe's loop sequence.

The simplicity and speed of this assay should allow routine use in areas where a parallel genetic test would be advantageous but is often too difficult or time-consuming. Examples of uses include genetic screening for human inherited diseases, determining identity or parentage, identifying pathogens and their antibiotic resistance capabilities, and screening foods or materials for contaminating organisms and viruses.

Other embodiments would include other labels, such as radioisotopic, isotopic, enzymatic, chromogenic, fluorescent, chemiluminescent, or primary and secondary antibodies that are either attached to the PRL during synthesis or added in a later step (before or after hybridization or restriction digestion have occurred). Many new methods for labeling, such as multiple labels, fluorescent beads, and microparticles, are currently being developed and may be used with PRLs in the future [24].

The PRLs can be attached to glass or silicon surfaces according to several other methods [25]. PRLs can also be attached to gold or other metal surfaces via sulfhydryl moieties and to microtiter wells via a biotin-streptavidin linkage [26].

Labels and immobilization moieties can be located anywhere along the length of the PRL, including the loop sequence region, providing that their location does not interfere with hybridization and that the restriction site lies between the surface-attachment site and the position of the label.

The PRL may have different types of spacers or multiple spacers for the purpose of raising the PRL above the surface; to reduce steric hindrances during the hybridization, washing, restriction enzyme digestion; etc. In addition, oligonucleotides can be constructed for use as controls that always remain or are always removed from the surface during the assay.

### LITERATURE CITED

1. W. Bains and G. C. Smith. J. Theor. Biol. 135:303-307 (1989).

2. W. Bains, Genomics 11:294-301 (1991).

3. E. M. Southern, U. Maskos, and J. K. Elder. Genomics 13:1008-1017 (1992).

4. U. Maskos and E. M. Southern. Nucleic Acids Res. 20:1679-1684 (1992).

5. S. P. A Fodor, J. L. Read, M. C. Pirrung, L. Stryer, A. T. Lu, and D. Solas. Science 251: 767-773 (1991).

6. K. R. Khrapko, Y. P. Lysov, A. A. Khorlin, I. B. Ivanov, G. M. Yershov, S. K. Vasilenko, V. L. Florentiev, and A. D. Mirzabekov. DNA Sequencing and Mapping 1:375-388 (1991).

7. M. D. Eggers, M. E. Hogan, R. K. Reich, J. B. Lamture, K. L. Beattie, M. A. Hollis, D. J. Ehrlich, B. B. Kosicki, J. M. Shumaker, R. S. Varma, B. E. Burke, A. Murphy, and D. D. Rathman. in Advances in DNA Sequencing Technology, SPIE 1891, (1993).

8. C. R. Cantor, A. Mirzabekov, and E. Southern. Genomics 13, 1378-1383 (1992).

9. S. Tyagi and F. R. Kramer, Nature Biotech. 14:303-308 (1996).

10. A. Piatek, S. Tyagi, A. Pol, A. Telenti, L. Miller, F. Kramer, and D. Alland, Nature Biotechnology 16:359-363 (1998).

11. A. Chenn, D. Dorfman, C. Walsh, and J. Winkelman, Amer. J. of Clinical Pathology 112:1 (1999).

12. B. Giesendorf, J. Vet, S. Tyagi, E. Mensink, F. Trijbels, and H. Blom, Clinical Chemistry 44(3):482-486 (1998).

13. I. Täpp, L. Malmberg, E., Rennel, M. Wik, and A.-C. Syvänen, Biotechniques 28:732-738 (2000).

14. D. L. Sokol, X. Zhang, P. Lu, and A. M. Gewirtz, Proc. Natl. Acad. Sci. U.S.A., 95:11538-11543 (1998).

15. T. Matsuo, Biochimica et Biophysica Acta, 1379:178-184 (1998).

16. S. Tyagi, “Detecting mRNAs in Living Cells with Molecular Beacons”, Contract R01ES10536, National Institutes of Health Environmental Genome Project.

17. X. Fang, X. Liu, S. Schuster, and W. Tan, J. Am Chem. Soc. 121(12):2921-2922 (1999).

18. L. J. Brown, J. Cummins, A. Hamilton, and T. Brown, J. Chem. Soc., Chem. Commun. 2000:621-622 (2000).

19. J. K. Actor, T. Kuffner, C. S. Dezzutti, R. L. Hunter, and J. M. McNicholl, J. Immunol.

Meth. 211:65-77 (1998).

20. Venetianer, P. and A. Kiss (1981) In: Gene Amplification and Analysis, Volume 1: Restriction Endonucleases, J. Chirikjian, ed. (Elsevier North Holland, Inc.) 209-215.

21. Blakesley, R. (1981) In: Gene Amplification and Analysis, Volume 1: Restriction Endonucleases, J. Chirikjian, ed. (Elsevier North Holland, Inc.) 1-34.

22. Sambrook, J., Fritsch, E., and T. Maniatis (1989) Molecular Cloning: A Laboratory Manual, 2nd Edition (Cold Spring Harbor Laboratory Press) 5.3-5.32.

23. Maldonado-Rodriguez, R., Espinosa-Lara, M., Loyola-Abitia, P., Beattie, W., and K. Beattie (1999) Molecular Biotechnology 11:13-25.

24. Kricka, L. (1999) Clinical Chemistry 45:453-458.

25. Subramanian, A., Kennel, S., Oden, P., Jacobson, K., Woodward, J., and M. Doktycz (1999) Enzyme and Microbial Technology 24:26-34.

26. Fritz, J., Baller, M., Lang, H., Rothuizen, H., Vettiger, P., Meyer, E., Guntherodt, H.-J., Gerber, C., and J. Gimzewski (2000) Science 288:316-318.

We claim:

1. A nucleic acid probe for detecting hybridization of unlabeled nucleic acid comprising:
an oligonucleotide having
(a) a surface-coupling group,
(b) a first sequence called the “loop sequence” that is complementary to a target nucleic acid sequence;
(c) a second and a third sequence, located on opposite ends of the loop sequence and which can hybridize to each other in the absence of the target nucleic acid sequence to form what is called a “stem structure,” wherein said stem structure contains a restriction enzyme cleavage site that is not present in the loop sequence (b) when hybridized to the target nucleic acid sequence; and
(d) a label located on the opposite side of the restriction site from the surface-coupling group such that cleavage by restriction enzyme specific for the restriction site of the stem sequences detaches the label from the surface;
wherein said loop sequence makes up all, or part, of the nucleotides between the complementary sequences, and wherein hybridization of the fully complementary target nucleic acid to the loop sequence breaks the intramolecular hybridization bonds of the stem structure and removes the restriction site.

2. The nucleic acid probe of claim 1, wherein the label is a bioluminescent, chemiluminescent, radioisotopic, isotopic, enzymatic, chromogenic, fluorescent or antibody label.

3. The nucleic acid probe of claim 2, wherein the bioluminescent label is luciferase, aequorin, alkaline phosphatase, horseradish peroxidase or β galactosidase.

4. The nucleic acid probe of claim 2, wherein the chemiluminescent label is acridinium.

5. The nucleic acid probe of claim 1, wherein the restriction enzyme recognition site is 4 to 8 nucleic acid bases in length.

6. The nucleic acid probe of claim 1, wherein the restriction enzyme site is specifically cleaved by a restriction enzyme selected from the group consisting of AaaI, AatI, AatII, AbeI, AbrI, Acc16I, Acc65I, AccB1I, AccB2I, AccBSI, AccEBI, AccI, AccII, AccIII, AceI, AceII, AciI, AcpI, AcrII, AcyI, AeuI, Afa16RI, Afa22MI, AfaI, AfeI, AflI, AflIII, AgeI, AhaB8I, AhaI, AhaII, AhyI, AitI, AjoI, AliAJI, AliI, AluI, Alw21I, Alw44I, Ama87I, AocI, AocII, Aor13HI, Aor51HI, AorI, AosI, AosII, ApaCI, ApaI, ApaLI, ApaORI, ApiI, ApyI, AquI, AscI, AseII, AsiAI, AsiI Bce751I, AsiSI, Asp10HI, Asp713I, Asp718I, Asp745I, AspAI, AspHI, AspLEI, AspMDI, AspMI, AspNI, AspS9I, AstWI, AsuC2I, AsuI, AsuII, AsuIII, AsuNHI, AvaI, AvaII, AvcI, AviII, AvrBII, AvrII, AxyI, Bac36I, Bal228I, BalI, BamHI, BamNxI, BanI, BanII, BavAI, BavAII, BavBI, BavBII, BavI, BbeI, Bbi24I, BbiII, BbrPI, BbuI, Bbv12I, BbvAIII, BbvBI, BbvCI, Bca77I, Bce22I, Bce243I, BceBI, BciBII, BcnI, Bco118I, Bco27I, BcoAI, BcoI, BcuAI, BdiSI, BecAII, BepI, BetI, BfaI, Bfi57I, Bfi89I, BfmI, Bim19I, Bim19II, BimI, BliHKI, BlnI, BloHI, BlpI, BluI, Bme12I, Bme1390I, Bme142I, Bme18I, Bme216I, Bme361I, BmyI, BnaI, Bpu10I, Bpu1102I, Bpu14I, Bpu95I, BpuB51I, BpuDI, BpuI, BsaAI, BsaHI, BsaJI, BsaOI, BsaWI, BscBI, BscFI, Bse118I, Bse15I, Bse16I, Bse17I, Bse1I, Bse1I, Bse21I, Bse24I, Bse634I, Bse64I, BseAI, BseBI, BseDI, BseGI, BseGI, BseNI, BseNI, BsePI, BseQI, BseSI, BseT10I, BseT9I, BseX3I, Bsh1236I, Bsh1285I, Bsh45I, BshFI, BshGI, BshI, BshKI, BshNI, BshTI, BsiCI, BsiEI, BsiHKAI, BsiI, BsiKI, BsiLI, BsiMI, BsiSI, BsiWI, BsiZI, BsmSI, BsoBI, BsoCI, BsoFI, Bsp105I, Bsp119I, Bsp120I, Bsp123I, Bsp1286I, Bsp13I, Bsp143I, Bsp143II, Bsp153AI, Bsp1720I, Bsp1894I, Bsp19I, Bsp2095I, Bsp211, Bsp4009I, Bsp50I, Bsp519I, Bsp63I, Bsp67I, Bsp68I, Bsp6I, Bsp98I, BspA2I, BspAAI, BspAAIII, BspAI, BspBI, BspBII, BspBRI, BspCI, BspEI, BspF4I, BspFI BspJI, BspKI, BspKT6I, BspLAI, BspLAII, BspLI, BspLS2I, BspM39I, BspMII, BspMKI, BspNI, BspO4I, BspR7I, BspRI, BspT104I, BspT107I, BsrAI, BsrBI, BsrFI, BsrI, BsrI, BsrSI, BsrSI, BssAI, BssECI, BssHI, BssHII, BssKI, BssSI, BssTII, Bst100I, Bst11I, Bst11I, Bst1I, Bst2BI, Bst2I, Bst2UI, Bst31NI, Bst38I, Bst40I, Bst4CI, BstACI, BstB7SI, BstBAI, BstBI, BstBZ153I, BstD102I, BstDEI, BstDSI, BstEII, BstENII, BstF5I, BstF5I, BstFNI, BstH2I, BstHHI, BstHZ55I, BstI, BstJZ301I, BstM6I, BstMCI, BstMZ611I, BstNI, BstNSI, BstOI, BstPI, BstSFI, BstSI, BstT10I, BstT9I, BstUI, BstVI, BstX2I, BstXI, BstYI, BstZI, Bsu1532I, Bsu1854I, Bsu23I, Bsu36I, Bsu54I, BsuBI, BsuFI, BsuRI, BteI, BtgI, BthAI, BthDI, BthEI, BtkI, BtkII, BtrI, BtsI, BvuBI, BvuI, Cac8I, CacI, CauB3I, CauI, CauII, CbiI, CboI, CbrI, CciNI, CcoI, CcrI, CcuI, CcyI, CelI, CelII, CflI, CfoI, Cfr10I, Cfr13I, Cfr42I, Cfr6I, Cfr9I, CfrA4I, CfrBI, CfrI, CfrJ4I, CfuII, ChaI, CltI, CpfI, CpoI, CscI, Csp45I, Csp68KI, Csp68KII, Csp68KVI, Csp6I, CspAI, CspBI, CspI, CspKVI, CstI, CthII, CviAI, CviAII, CviBI, CviJI, CviQI, CvIRI, CviRII, CviTI, CvnI, DdeI, DmaI, DpnII, DraII, DsaI, DsaII, DsaIII, DsaIV, DsaV, Eae46I, EaeAI, EaeI, EagBI, EagI, EagMI, EcaI, Eci125I, EciI, Ecl136II, Ecl18kI, Ecl2zI, Ecl37kI, EclRI, EclXI, Eco130I, Eco137kI, Eco13kI, Eco147I, Eco1831I, Eco21kI, Eco24I, Eco27kI, Eco29kI, Eco47I, Eco47III, Eco52I, Eco53kI, Eco56I, Eco64I, Eco72I, Eco75KI, Eco78I, Eco81I, Eco88I, Eco91I, EcoHI, EcoHK31I, EcoICRI, EcoO109I, EcoO128I, EcoO65I, EcoRII, EcoT14I, EcoT38I, EgeI, EheI, ErhB9I, ErhB9II, ErhI, ErpI, EspI, FauBII, FblI, FbrI, FdiI, FdiII, FgoI, FmuI, Fnu4HI, FnuAI, FnuCI, FnuDI, FnuDII, FnuDIII, FnuEI, FriOI, FseI, Fsp1604I, Fsp4HI, FspI, FspII, FspMSI, FssI, FunI, GalI, GceGLI, GceI, GdiI, GdiII, HacI, HaeI, HaeII, HaeIII, HalII, HapII, HgiAI, HgiBI, HgiCI, HgiCII, HgiCIII, HgiDI, HgiDII, HgiEI, HgiGI, HgiHI, HgiHII, HgiHIII, HgiI, HgiJI, HgiJII, HgiS22I, HhaI, HhaII, Hin1I, Hin1II, Hin2I, Hin6I, HincII, HindII, HinfI, HinJCI, HinP1I, HpaII, Hpy178III, Hpy188I, Hpy188III, Hpy51I, Hpy8I, Hpy99I, HpyBI, HpyBII, HpyCH4I, HpyCH4III, HpyCH4IV, HpyCH4V, HsoI, Hsp92I, Hsp92II, HspAI, ItaI, KasI, Kaz48kI, KoxII, Kpn2I, Kpn2kI, Kpn378I, Kpn49kII, KpnI, KspI, Kzo49I, Kzo9I, LlaAI, LlaBI, Lmu60I, LpnI, LspI, MabI, MaeI, MaeII, MaeIII, MaeK81I, MaeK81II, MavI, MbiI, MboI, MchAI, MchAII, MchI, McrI, MflI, MfoAI, Mgl14481I, MgoI, MhaAI, MhlI, MkrAI, MlaAI, MlaI, MlsI, MltI, Mlu23I, Mlu31I, MluB2I, MluI, MluNI, Mly113I, MnoI, MroI, MroNI, MscI, Msp17I, Msp67I, MspA1I, MspB4I, MspI, MspR9I, MspV281I, MspYI, MstI, MstII, MthZI, MvaI, MvnI, MvrI, MxaI, NaeI, NarI, NblI, NciI, NcoI, NdaI, NdeII, NgoAIII, NgoAIV, NgoMIV, NgoPII, NgoPIII, NheI, NlaII, NlaIII, NlaIV, Nli3877I, NmeCI, NmeRI, NmuCI, NopI, NotI, NphI, NruI, NsbI, Nsp29132II, Nsp7121I, NspBII, NspHI, NspI, NspII, NspIII, NspIV, NspLKI, NspSAI, NspSAII, NspSAIV, NspV, NunII, OkrAI, OxaNI, Pac25I, Pae14kI, Pae17kI, Pae5kI, PaeAI, PaeBI, PaeHI, PaeI, PaePI, PaeQI, PaeR7I, PalI, PamI, PamII, PanI, PauAI, PauI, Pde12I, Pde133I, Pde137I, PdiI, PfaAI, PfaAIII, Pfl21I, Pfl23II, Pfl27I, Pfl8I, PflKI, PinAI, PinBII, PlaAI, PlaAII, PlaI, PlaII, Ple19I Psu161I, PmaCI, Pme55I, PmiI, PpaAI, PpaAII, PpeI, PpuAI, PpuMI, PpuXI, Psp03I, Psp124BI, Psp23I, Psp5II, PspAI, PspALI, PspEI, PspGI, PspLI, PspN4I, PspOMI, PspPI, PspPPI, PssI, PstI, PstNHI, PsuAI, PsuI, PtaI, PunAI, PunAII, Pvu84II, PvuI, PvuII, RalF40I, RflFI, RmaI, RsaI, RshI, RspLKI, RspLKII, Rsr2I, RsrII, Rtr63I, RtrI, SacI, SacII, SacNI, SalI, SalPI, SanDI, SarI, SatI, Sau3239I, Sau3AI, Sau96I, SauBMKI, SauHPI, SauI, SauLPI, SauLPII, SauMI, SauNI, SauSI, SbfI, Sbi68I, Sbo13I, SbvI, SceIII, SchZI, SciI, SciNI, ScrFI, SdaI, SduI, SecI, SelI, SenPT14bI, SenPT16I, SexAI, SexBI, SexCI, SfaI, SfcI, SfeI, SflI, SfoI, Sfr274I, Sfr303I, SfuI, SgfI, SgrAI, SgrBI, SimI, SinI, SlaI, SleI, Slu1777I, SmaI, SmlI, SmuEI, SniI, SnoI, Sol10179I, SolI, SpaHI, SphI, SplI, SpoI, SpuI, SrfI, SrlI, Sru30DI, SscL1I, Sse1825I, Sse232I, Sse8387I, SseAI, SseBI, SshAI, SslI, SsoII, Ssp1I, Ssp5230I, SspAI, SspI, SspRFI, SstI, SstII, SteI, Sth1171I, Sth134I, Sth368I, SthI, StrI, StuI, StyD4I, StyI, SuaI, SunI, SurI, SviI, TaaI, TaiI, Taq52I, TaqI, TaqXI, TauI, ThaI, TliI, Tru201I, TscI, TseI, Tsp1I, Tsp1I, Tsp32I, Tsp32II, Tsp45I, Tsp49I, Tsp4CI, TspBI, TthHB8I, UnbI, Uur960I, VneI, VpaK11AI, VpaK11BI, XceI, XciI, XcyI, XhoI, XhoII, XmaCI, XmaI, XmaIII, XmaJI, XmiI, XorII, XpaI, XspI, YenI, and ZanI.

7. The nucleic acid probe of claim 1, wherein the surface coupling group is a covalent coupling group.

8. The nucleic acid probe of claim 1, wherein the label is attached to the oligonucleotide through a biotin-steptavidin coupling.

9. The nucleic acid probe of claim 1, wherein the oligonucleotide contains spacer groups at either or both ends.

10. The nucleic acid probe of claim 9, wherein the spacer groups are polythymine spacers.

11. The nucleic acid probe of claim 7, wherein the covalent surface coupling group is an aminopropanol moiety.

12. The nucleic acid probe of claim 7, wherein the oligonucleotide is attached to a surface with the covalent coupling group.

13. The nucleic acid probe of claim 12, wherein the surface is a glass, silicon, gold, metal or plastic surface.

14. The nucleotide probe of claim 1, wherein the loop sequence is 16 to 25 nucleotides in length.

15. A method of determining the presence of a target nucleic acid comprising:
exposing a sample to a nucleic acid probe of claim 1 under effective conditions for specific hybridization of the target nucleic acid to the nucleic acid probe;
adding restriction enzyme specific for the restriction site contained in the stem structure and under conditions to cleave the nucleic acid probe with the restriction enzyme;
determining the presence of the target nucleic acid, wherein the presence or amount of label is indicative of the presence of target nucleic acid.
